(19) United States
(12) Patent Application Publication
(10) Pub. No.: US 2025/0354141 A1
(43) Pub. Date: Nov. 20, 2025

(54) TARGETING ARHGAP5 GENE FUNCTION IN CANCER WITH ARHGAP35 ALTERATION

(71) Applicant: The Children's Medical Center Corporation, Boston, MA (US)

(72) Inventor: Steen H. Hansen, Brookline, MA (US)

(73) Assignee: The Children's Medical Center Corporation, Boston, MA (US)

(21) Appl. No.: 18/866,473

(22) PCT Filed: May 17, 2023
(86) PCT No.: PCT/US2023/022533

§ 371 Date: Nov. 15, 2024

Related U.S. Application Data
(60) Provisional application No. 63/343,207, filed on May 18, 2022.

(51) Int. Cl. C12N 15/113; A61P 35/00; C12N 15/86; C12Q 1/6869; C12Q 1/6886

(52) U.S. Cl. CPC C12N 15/113; A61P 35/00; C12N 15/86; C12Q 1/6869; C12Q 1/6886; C12N 2310/11; C12N 2310/14; C12N 2310/531; C12N 2740/15043; C12N 2750/14143; C12Q 2600/106

(57) ABSTRACT

Provided herein are methods for treating cancers that have low or no expression or activity of ARHGAP35, also referred to as p190A, by administering an effective amount of a therapeutic agent that decreases the expression or activity of ARHGAP5, also referred to as p190B. As demonstrated herein, administration of an agent that decreases expression or activity of ARHGAP5 can cause paralog lethality in a cancer having low or no expression or activity of ARHGAP35, leading to clearance of the cancer in a subject.

### RELATED APPLICATION

This application claims the benefit under 35 U.S.C. § 119 (e) to U.S. Provisional Application No. 63/343,207, entitled “TARGETING ARHGAP5 GENE FUNCTION IN CANCER WITH ARHGAP35 ALTERATION,” filed on May 18, 2022, the entire contents of which are incorporated herein by reference.

### GOVERNMENT SUPPORT

This invention was made with government support under R01-CA205158 awarded by the National Institutes of Health. The government has certain rights in the invention.

### BACKGROUND

ARHGAP35, a gene on chromosome 19 that encodes p190A RhoGAP (p190A), an enzyme that promotes GTP hydrolysis on Rho and Rac GTPases, is among the most frequently mutated genes in human cancer. Alterations in ARHGAP35 occur in approximately 4% of human cancers and occur with frequency across a broad range of cancer types. For this reason, ARHGAP35 has been regarded as a “pan-cancer” gene. Although mutations in ARHGAP35 are not thought to be oncogenic in and of their own, ARHGAP35 alterations are observed to be enriched in oncogene-negative cancers, for which targeted therapies generally do not exist. However, despite the apparent correlation between cancer and alteration of ARHGAP35, thus far no study has been able to determine a functional role for ARHGAP35 or its paralog, ARHGAP5 (p190B), in the development of human cancers, beyond that of a general tumor suppressor.

### SUMMARY

Some aspects of the present disclosure relate to methods of treating a cancer in a subject. In some aspects, such a method comprises administering to a subject in need thereof an effective amount of an agent that results in a decrease in the expression or activity of ARHGAP5, wherein the cancer is a cancer in which ARHGAP35 expression or activity is decreased.

In some embodiments, the agent inhibits expression of ARHGAP5 in the cancer. In some embodiments, the agent comprises a small interfering RNA (siRNA), a short hairpin RNA (shRNA), or an antisense oligonucleotide (ASO) that is complementary to an ARHGAP5 mRNA expressed in the cancer. In some embodiments, the agent comprises a viral vector that encodes a siRNA or a shRNA that is complementary to an ARHGAP5 mRNA expressed in the cancer. In some embodiments, the viral vector is a lentiviral vector or a recombinant adeno-associated viral (rAAV) vector. In some embodiments, the siRNA, shRNA, or ASO is at least 60%, at least 70%, at least 80%, at least 90%, at least 95%, at least 99%, or 100% complementary to a region of an ARHGAP5 mRNA expressed in the cancer.

In some embodiments, the agent inhibits the activity and/or stability of a p190B protein translated from an ARHGAP5 mRNA expressed in the cancer. In some embodiments, the agent comprises a small molecular inhibitor.

In some embodiments, the cancer is a cancer in which ARHGAP35 mRNA expression is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue. In some embodiments, the cancer is a cancer in which activity of a p190A protein translated from an ARHGAP35 mRNA is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue.

In some embodiments, the cancer comprises at least one alteration of ARHGAP35. In some embodiments, the alteration of ARHGAP35 is a mutation in a gene encoding ARHGAP35. In some embodiments, the alteration of ARHGAP35 is a deletion of a gene encoding ARHGAP35. In some embodiments, the alteration of ARHGAP35 is an epigenetic modification in a gene encoding ARHGAP35.

In some embodiments, the cancer is an oncogene-negative cancer. In some embodiments, the cancer is a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer. In some embodiments, the cancer is selected from endometrial carcinoma, uterine carcinosarcoma, colon adenocarcinoma, lung squamous carcinoma, bladder cancer, cervical carcinoma, or stomach cancer. In some embodiments, the cancer is a metastatic cancer.

In some embodiments, the Salvador-Warts-Hippo (SWH) pathway is inactivated in the cancer. In some embodiments, the level of activated Yes-associated protein (YAP) and/or Transcriptional coactivator with PDZ-binding motif (TAZ) is increased in the cancer, as compared to non-cancerous tissue. In some embodiments, the level of activated Rho (Rho-GTP) is increased in the cancer, as compared to non-cancerous tissue. In some embodiments, contact inhibition of cell proliferation (CIP) activity is decreased in the cancer, as compared to non-cancerous tissue.

In some embodiments, the subject is a mammal. In some embodiments, the subject is a human.

In some embodiments, the method further comprises determining if the cancer comprises one or more alterations of ARHGAP35 prior to administration of the agent. In some embodiments, the determination comprises collecting a sample from the subject, sequencing the genome of one or more cancer cells present in the sample, and determining the presence of one or more alterations of ARHGAP35 in the cancer. In some embodiments, the sample comprises a tissue biopsy, blood sample, a serum sample, a plasma sample, a saliva sample, a sputum sample, a urine sample, a fecal sample, a lymphatic fluid sample, a synovial fluid sample, a cerebrospinal fluid sample, or an interstitial fluid sample. In some embodiments, the tissue biopsy is a tumor biopsy. In some embodiments, the one or more alterations of ARHGAP35 comprise a mutation in a gene encoding ARHGAP35. In some embodiments, the one or more alterations of ARHGAP35 comprise a deletion of a gene encoding ARHGAP35. In some embodiments, the one or more alterations of ARHGAP35 comprise an epigenetic modification in a gene encoding ARHGAP35.

In some embodiments, the administration occurs systemically or locally. In some embodiments, the administration occurs via injection. In some embodiments, the injection is intravenous or intratumoral injection. In some embodiments, the administration occurs more than once. In some embodiments, the administration occurs between once per week and once per six months.

In some embodiments, the administration results in an increase in SWH pathway activation in the cancer, as compared to SWH pathway activation in the cancer prior to the administration. In some embodiments, the administration results in a decrease in the level of activated YAP and/or TAZ in the cancer, as compared to the level of activated YAP and/or TAZ in the cancer prior to the administration. In some embodiments, the administration results in an increase of CIP activity in the cancer, as compared to CIP activity in the cancer prior to the administration.

In some embodiments, the administration results in clearance of the cancer in the subject. In some embodiments, the administration results in clearance of up to 10%, up to 20%, up to 30%, up to 40%, up to 50%, up to 60%, up to 70%, up to 80%, up to 90%, up to 95%, up to 99%, or 100% of the cancer in the subject.

In further aspects, the present disclosure relates to methods of treating a cancer in a subject, comprising administering to a subject in need thereof an effective amount of an agent that results in a decrease in the expression or activity of ARHGAP35, wherein the cancer is a cancer in which ARHGAP5 expression or activity is decreased.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C show that co-depletion of p190A and p190B from Madin-Darby canine kidney (MDCK) cells activates YAP-regulated gene transcription that perturbs contact inhibition of cell proliferation (CIP). FIG. 1A shows that expression of a dominant negative YAP5SA+S94A restores CIP in p190A/p190B depleted MDCK cells grown in Transwells. FIG. 1B shows transcriptome analysis of p190A/p190B knockdown cells relative to control cells. FIG. 1C shows quantification of YAP-target gene expression as determined by quantitative polymerase chain reaction (qPCR).

FIGS. 2A-2C show that exogenous Myc-tagged p190A represses YAP activity and restores CIP in cells depleted of p190A and p190B in a LATS kinase-dependent manner. FIG. 2A shows immunoblotting of whole cell lysates to detect relevant protein levels. FIG. 2B shows quantification of YAP-target gene levels as determined by qPCR. FIG. 2C shows confocal imaging of control and p190A/p190B knockdown cells expressing either Myc-p190A or HA-LATS2-KR, a dominant negative variant of LATS2 kinase. Cytosolic fluorescence of cells expressing HA-LATS2-KR is due to the expression of HA-LATS2-KR from a bi-cistronic vector with enhanced green fluorescent protein (eGFP).

FIGS. 3A and 3B show that individual knockdown of p190A or p190B is sufficient to activate YAP and perturb CIP in MDCK cells. FIG. 3A shows that YAP translocates into nuclei of cells with depletion of p190A and/or p190B, as shown in optical sections of peripheral cells of MDCK cell spheroids cultured in Matrigel. FIG. 3B shows that MDCK spheroids with p190A and/or p190B knockdown retain polarity towards rudimentary lumens, as shown in optical sections through the center of MDCK cell spheroids.

FIGS. 4A-4I show that restoring p190A expression in non-small cell lung cancer (NSCLC) cells with ARHGAP35 alteration activates LATS kinases, elicits MET, and promotes CIP via induced E-cadherin expression. FIG. 4A shows immunoblotting of whole cell lysates from control and H661-p190A cells. FIG. 4B shows growth curves of control cells and H661-p190A cells. 5×10⁴ cells were seeded per well of a 6-well plate and propagation for the number of indicated days. Cell media was replaced every 2 days. FIG. 4C shows immunoblotting of whole cell lysates to detect markers of Hippo pathway activation, as well as cyclin A. FIG. 4D shows quantification of cyclin A immunoblotting in FIG. 4C by densitometry. ***: p<0.01 by Student's t test. FIG. 4E shows mRNA transcript levels of YAP target genes CTGF and CYR61 as determined by qPCR. ***: p<0.01 by Student's t test. FIG. 4F shows that p190A restores cell growth in monolayers a LATS1/2-dependent manner. FIG. 4G shows mRNA transcript levels of TWIST1, SNAI2, and ZEBI as determined by qPCR. ***: p<0.01 by Student's t test. FIG. 4H shows that p190A elicits the expression of a cassette of epithelial genes by an E-cadherin dependent mechanism, as determined by qPCR. FIG. 4I shows a model summarizing the tumor suppressor function of p190A. Reconstitution of NSCLC cells with p190A activates the Hippo pathway, which induces expression of CDH1. E-cadherin subsequently enhances activation of LATS kinases.

FIGS. 5A-5C show that p190A suppresses tumorigenesis in severe combined immunodeficient (SCID) mice injected with H661-p190A cells. FIG. 5A shows mice injected with control or H661-p190A cells at 3 weeks. Tumor size in the control mouse necessitated euthanasia at 4.5 weeks, however no tumor tissue was observed in the H661-p190A mice at 20 weeks and beyond. FIG. 5B shows quantification of xenograft tumor volume in mice injected with control or H661-p190A cells. Data are presented as means±standard deviation (SD). *: p<0.05 by Student's t test. FIG. 5C shows Kaplan-Meier survival plot for mice injected with control or H661-p190A cells. Mice were euthanized when a tumor exceeded 8 mm along the longest axis. **: p<0.025 by log-rank test.

FIGS. 6A-6D show an analysis of naturally occurring ARHGAP35 mutations in human cancer. FIG. 6A shows immunoblotting of whole cell lysates to detect wild type and mutant p190A forms, LATS activation, E-cadherin, and cyclin A. FIG. 6B shows quantification of transcript levels for YAP target genes CTGF and CYR61, as well as CDH1 encoding E-cadherin. FIG. 6C shows a principal component analysis (PCA) plot illustrating that E400K and R1284W ARHGAP35 mutations exhibit complete loss of function in respect to gene regulation. FIG. 6D shows immunofluorescence of HeLa cells expressing p190A forms harboring recurrent cancer mutations. E400K and S886Y mutant forms retain full GAP activity, while the R1284W mutant, which targets the arginine finger of the GAP domain, exhibits complete loss of function. The S229L mutation, which impacts the GTPase domain of p190A known to modulate GAP activity displays an intermediate phenotype.

FIG. 7 shows a schematic illustrating the principle of paralog lethality. Two paralogs can arise from a single ancestral gene and retain overlapping functions. Although cells may be viable when one paralog or the other is knocked down, ablation of both paralogs simultaneously may cause cell death.

FIGS. 8A-8F shows that canine orthologs of ARHGAP35 and ARHGAP5 exhibit paralog lethality. FIG. 8A shows a scrape wounding assay with MDCK cells depleted of p190A and/or p190B. Cells expressing p190A or p190B grow to close the wound within 48 hours, however cells lacking both p190A and p190B expression do not. FIG. 8B shows quantification of data shown in FIG. 8A. FIG. 8C shows proliferation of MDCK cells with knockdown of p190A and/or p190B. Cells lacking both p190A and p190B exhibit a severe growth defect. FIG. 8D shows immunoblotting of whole cell lysates from an anoikis assay conducted with MDCK cells depleted of p190A and/or p190B. The asterisk (*) marks the smaller fragment of cleaved PARP, indicating increased cell death in cells with both p190A and p190B knockdown. FIG. 8E shows quantification of tumor size in mice inoculated with MDCK cells expressing the B-cell lymphoma 2 (BCL2) oncogene with or without knockdown of p190A and p190B. p190A/p190B knockdown prevented growth of BCL2-expressing MDCK tumors in mice. FIG. 8F shows a Kaplan-Meier plot for mice in FIG. 8E. Mice were euthanized when tumor volume exceeded 250 μm³.

FIGS. 9A-9G show schematics of experimental workflows for assessing paralog lethality of ARHGAP35 and ARHGAP5. FIG. 9A shows a schematic of a co-culture assay for assessing paralog lethality of ARHGAP35 and ARHGAP5. Control MDCK cells are cultured with MDCK cells transformed by one or more major oncogenes (MDCK^{onc}), either in the presence or absence of p190A and/or p190B knockdown. FIG. 9B shows a schematic of a co-culture assay for assessing paralog lethality of ARHGAP35 and ARHGAP5. Primary human bronchial epithelial cells (HBEC) control cells are cultured with NSCLC cells that either express wild-type p190A or lack p190A (p190A^{-/-}), in the presence or absence of p190B knockdown. FIG. 9C shows a schematic of an anoikis assay for assessing paralog lethality of ARHGAP35 and ARHGAP5. MDCK control cells are cultured on a slide with MDCK^{one} cells in the presence or absence of p190A/p190B knockdown. FIG. 9D shows a schematic of an in vivo assay using a xenograft mouse model with MDCK^{onc}. Mice are injected with either MDCK^{one} cells, MDCK^{one} cells with doxycycline-inducible p190A knockdown, or MDCK^{onc} cells with doxycycline-inducible p190A and p190B knockdown. Xenograft model mice are optionally fed doxycycline chow to induce knockdown of p190A and/or p190B. FIG. 9E shows a schematic of an in vivo assay for assessing paralog lethality of ARHGAP35 and ARHGAP5 using a xenograft mouse model with H661 NSCLC harboring a known ARHGAP35 mutation. Mice are injected with either H661 control cells, H661 cells with doxycycline-inducible p190B knockdown, or H661 p190A control cells. Xenograft model mice are optionally fed doxycycline chow to induce knockdown of p190B in NSCLC cells harboring the ARHGAP35 mutation. FIG. 9F shows a schematic of an in vivo assay for assessing paralog lethality of ARHGAP35 and ARHGAP5 using a xenograft mouse model with NSCLC with or without a known ARHGAP35 mutation. Mice are injected with NSCLC cells harboring either wild-type ARHGAP35 or mutant ARHGAP35 and doxycycline inducible p190B knockdown. Xenograft model mice are optionally fed doxycycline chow to induce knockdown of p190B in NSCLC cells. FIG. 9G shows a schematic of an in vivo assay for assessing paralog lethality of ARHGAP35 and ARHGAP5 using a xenograft mouse model with NSCLC with or without a known ARHGAP35 mutation. Injected mice are subsequently administered either a mock viral vector or a viral vector that induces knockdown of p190B in NSCLC cells.

### DETAILED DESCRIPTION OF CERTAIN EMBODIMENTS

Aspects of the present disclosure are based on the discovery of paralog lethality between Rho GTPase Activating Protein 35 (ARHGAP35) and Rho GTPase Activating Protein 5 (ARHGAP5), which encode the Rho GTPase-activating proteins (RhoGAPs) p190A and p190B, respectively. Without wishing to be bound by theory, “paralog lethality” occurs when a cell requires the function of at least one of a set of paralogs with overlapping function to survive. Paralogs exhibiting paralog lethality can occur as the result of a gene duplication event in an essential gene, thereby producing one or more additional copies that retain the conserved function of the original gene. This phenomenon is referred to as “paralog buffering.” In principle, the redundancy of paralog buffering protects species from subsequent cellular stress or genetic alterations, because function of only one paralog of a paralog set is required for survival. However, loss of function of every required paralog in the set is lethal and may cause cells to undergo apoptosis.

Paralog lethality may have therapeutic applications, particularly for selectively targeting and killing cancer cells. Cancers arise due to genetic alterations that evade cellular DNA repair pathways and include genetic polymorphisms and/or deletions of parts of the genome that impair the regular function of genes. When these alterations negatively impact the function of tumor suppressor genes that prevent cancerous transformation by tightly regulating core cellular functions, such as, for example, cell division and programed cell death, cells may begin to grow uncontrollably and become tumorigenic. However, while alterations in tumor suppressor genes can promote tumorigenesis in this way, some tumor suppressor genes may be paralogs that function with paralog buffering. Therefore, while cancer cells may display enhanced tumorigenicity resulting from an alteration in one tumor suppressor gene belonging to a set of paralogs with overlapping function, these same cancer cells may in fact rely on the function of another tumor suppressor gene belonging to the same paralog set for survival. As such, perturbation of the remaining tumor suppressor gene(s) of the paralog set, for example, by administering one or more agents that inhibit function of the remaining paralog(s), would be lethal to the cancer. However, this perturbation would not impact the viability of healthy, non-cancerous cells, which lack alteration in the genes of the required paralog set.

### ARHGAP35 and ARHGAP5 as Targets for Cancer Treatment

As described in the Examples provided herein, ARHGAP35 and ARHGAP5 are identified as paralogs exhibiting paralog lethality. This phenomenon may be utilized, for example, to target cancer cells that have an alteration in ARHGAP35.

Without wishing to be bound by theory, ARHGAP35 and ARHGAP5 encode the proteins p190A (also referred to as GRF-1, GRLF1, or RhoGAP35) and p190B (also referred to as GFI2 or RhoGAP5), respectively. As used herein, the terms “ARHGAP35” and “p190A”, and the terms “ARHGAP5” and “p190B” may be used interchangeably, however, the terms “ARHGAP35” and “ARHGAP5” are generally used to refer to genes, while “p190A” and “p190B” are generally used to refer to the protein products encoded by these genes. p190A and p190B each function as RhoGAP proteins (also referred to as regulators of G protein signaling (RGS) proteins) that promote guanosine-5′-triphosphate (GTP) hydrolysis of Rho and Rac GTPases, such as, for example, RhoA and Rac1 GTPases. Rho and Rac GTPases regulate a plethora of cellular functions via cell signaling pathways, including, but not limited to, regulation of cell division (mitosis), cell polarity, cell morphology, and, in appropriate cell types, cell locomotion and phagocytosis. Hydrolysis of GTP bound to Rho and Rac GTPases deactivates signaling of these GTPases to downstream effectors. In turn, guanine nucleotide exchange factors (GEFs) act to exchange guanosine-5′-diphosphate (GDP) bound to Rho and Rac GTPases for GTP, thereby reactivating Rho and Rac signaling. In this way, Rho and Rac GTPases, RhoGAP proteins such as p190A and p190B, and GEFs dynamically control cell signaling, activating or deactivating signaling in response to a variety of internal and external stimuli.

p190A and p190B are each large, multifunction proteins. p190A is a 1499 amino acid protein which comprises a C-terminal GAP domain, which mediates interaction with Rho/Rac GTPases, as well as a pair of pseudo-GAP domains (psGAP1 and psGAP2), four FF motifs, and an N-terminal RAS-like domain. Many of these domains have scaffolding functions that are separate from the activity of the GAP domain. Specifically, the FF motifs and pseudo-GAP domains together mediate scaffolding of Rnd proteins (e.g., Rnd1), a subclass of Rho family GTPases that lack intrinsic GTPase activity and instead function as small signaling G proteins that antagonize Rho GTPases. Additionally, the FF motifs mediate scaffolding of the ubiquitously expressed transcription factor TFII-I (also referred to as SPIN and BAP135), thereby sequestering it in the cytoplasm. p190A further contains many sequences of unknown function. p190B is overall similar in size and function, measuring 1502 amino acids in size and comprising both a C-terminal GAP domain and four FF motifs, however, it shares only about 51% amino acid identity with p190A (see, e.g., NCBI Reference Sequences: NP_004482.4 and NP_001025226.1). Therefore, p190A and p190B are best understood as distinct paralogs that share common but not entirely identical functions.

Alterations in ARHGAP35 are among the most frequently occurring alterations in cancer, although ARHGAP35 alteration is not known to be in and of itself oncogenic. These alterations may result in the translation of an p190A isoform with decreased, if any, GAP activity, or may result in no p190A protein at all (e.g., occurring as a result of deletion of the ARHGAP35 locus). Genome-wide association studies (GWAS) have demonstrated that despite being frequently altered in human cancer, ARHGAP35 alterations are not specifically distributed among particular cancer types. ARHGAP35 alterations are also among the most commonly occurring alterations in oncogene negative cancers (cancers for which expression of an established oncogene cannot be determined), including, for example, oncogene negative lung cancers (e.g., non-small cell lung cancers (NSCLC)).

The precise role of ARHGAP35 in tumor suppression is not previously know. However, as explained in the Examples provided herein, ARHGAP35 and ARHGAP5 together function to repress the proto-oncogenic transcriptional co-activator Yes-associated protein (YAP), as well as its paralog, transcriptional coactivator with PDZ-binding motif (TAZ). By repressing YAP/TAZ, ARHGAP35 and ARHGAP5 function is demonstrated to activate Salvador-Warts-Hippo (SWH) pathway signaling and promote contact inhibition of cell proliferation (CIP), a process that prevents the proliferation of cells (e.g., epithelial cells) that are in contact with one another. Disruption of CIP, for example, by a reduction of expression of ARHGAP35 and ARHGAP5, or reduced activity of p190A or p190B, can drive malignant transformation as a result of unregulated cell division. Recognition of these mechanisms by which ARHGAP35 and ARHGAP5 prevent the development of cancer allows for these mechanisms to be utilized for the purpose of treating or preventing cancer in a subject (e.g., in a human patient). Specifically, these mechanisms may be utilized to treat or prevent cancers that are characterized by an alteration in ARHGAP35 resulting in reduced p190A function, especially oncogene-negative cancers for which no other specific and effective treatments are known.

### Administration of Agents to Induce ARHGAP35/ARHGAP5 Paralog Lethality in Cancer

In some aspects, the present disclosure provides for methods of inducing ARHGAP35/ARHGAP5 paralog lethality in cells of a subject, specifically in cancer cells of a subject. Generally, the methods disclosed herein are applicable for treating a cancer in a subject by administering an effective amount of an agent that results in a decrease in the expression or activity of ARHGAP5, wherein the cancer is a cancer in which ARHGAP35 expression or activity is decreased (e.g., as a result of an alteration in ARHGAP35). However, those of ordinary skill in the art will readily recognize that the methods described herein are also suitable for the treatment of a cancer in which ARHGAP5 expression or activity is determined to be decreased (e.g., as a result of an alteration in ARHGAP5), by administering an effective amount of an agent that results in a decrease in the expression or activity of ARHGAP35.

As used herein, the terms “administer,” “administering,” or “administration” refer to implanting, absorbing, ingesting, injecting, inhaling, or otherwise introducing an agent described herein, or a composition thereof (e.g., a pharmaceutical composition), in or on a subject. As used herein, the term “treatment,” “treat,” and “treating” refers to the application or administration of an agent described herein, or a composition thereof (e.g., a pharmaceutical composition), to a subject in need thereof for the purpose of reducing the severity of a disease (e.g., a cancer) in the subject. A “subject in need thereof” refers to an individual that has a disease, a symptom of the disease, or a predisposition toward the disease (e.g., a cancer). A method for treating a disease may encompass administering to a subject an agent described herein, or a composition thereof (e.g., a pharmaceutical composition) with the intention to cure, heal, alleviate, relieve, alter, remedy, ameliorate, improve, or affect the disease, a symptom of the disease, or predisposition toward the disease in the subject. A method for treating a disease may encompass prophylaxis, wherein an agent is administered to the subject for the purpose of preventing development of the disease, for example, in a subject that is not known to have the disease, but may develop or be at risk of developing the disease in the future.

As used herein, a “therapeutically effective amount” or “effective amount” refers to the amount of an agent that is sufficient to elicit the desired biological response in the subject, for example, alleviating one or more symptoms of the disease (e.g., a cancer). A therapeutically effective amount may be an amount that is either administered to the subject alone or in combination with one or more other agents. Effective amounts vary, as recognized by those skilled in the art, depending on such factors as the desired biological endpoint, the pharmacokinetics of the administered agent, the particular condition or disease being treated, the severity of the condition or disease, the individual parameters of the subject, including age, physical condition, size, gender and weight, the duration of the treatment, the nature of any other concurrent therapy, the specific route of administration, and like factors that are within the knowledge and expertise of the health practitioner to determine. These factors are well known to those of ordinary skill in the art and can be addressed with no more than routine experimentation. It is generally preferred that a maximum dose of the individual agents described herein or any combinations thereof to be used is at most the highest dose that can be safely administered to the subject according to sound medical judgment. Preferably, an effective dose is lower than the highest dose that can be safely administered to the subject. It will be understood by those of ordinary skill in the art, however, that a subject or health practitioner may select a lower dose (e.g., the minimum effective dose) in order to mitigate any potential risks of treatment, such as side effects of the treatment.

In some embodiments, for an adult subject of normal weight, doses ranging from about 0.01 to 1000 mg/kg of an agent may be administered. In some embodiments, the dose is between 1 to 200 mg. The particular dosage regimen, i.e., the dose, timing, and repetition, will depend on the particular subject and that subject's medical history, as well as the properties of the agent (such as the pharmacokinetics of the agent) and other consideration well known in the art.

Treating a disease (e.g., a cancer) may include delaying the development or progression of the disease or reducing disease severity. Treating the disease does not necessarily require curative results. As used herein, “delaying” the development of a disease means to defer, hinder, slow, retard, stabilize, and/or postpone progression of the disease in a subject. Delaying the progression of a disease may include delaying or preventing the spread of a disease occurring in a subject. This delay can be of varying lengths of time, depending on the history of the disease and/or individuals being treated. A method that delays the development of a disease, or delays the onset of the disease, is a method that reduces probability of developing one or more symptoms of the disease in a given time frame and/or reduces extent of the symptoms in a given time frame, as compared to the absence of such a method. Comparisons are typically based on clinical studies, using a number of subjects sufficient to give a statistically significant result.

The “development” or “progression” of a disease (e.g., a cancer) refers to initial manifestations and/or ensuing progression of the disease in a subject. Development of a disease can be detectable and assessed using standard clinical techniques as well known in the art. However, development also refers to progression that may be undetectable. For purpose of this disclosure, development or progression may refer to the development or progression of symptoms of a disease (e.g., a cancer). The development or progression of a disease may also refer to the spread of the disease to one or more tissues or organs of the subject not previously affected by the disease (e.g., metastasis of a cancer). The term “development” includes the occurrence, recurrence, and onset of a disease. As used herein “onset” or “occurrence” of a disease includes the initial onset of a disease, as well as recurrence of the disease (i.e., in a subject who has had the disease previously).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or a non-human animal. In some embodiments, the non-human animal is a mammal (e.g., rodent, e.g., mouse or rat), a primate (e.g., cynomolgus monkey or rhesus monkey), a commercially relevant mammal (e.g., cattle, pig, horse, sheep, goat, cat, or dog), or a bird (e.g., commercially relevant bird, such as chicken, duck, goose, or turkey). The non-human animal may be a male or female at any stage of development and may be a juvenile animal or an adult animal. The non-human animal may be a transgenic animal or genetically engineered animal.

In some embodiments, the subject is a companion animal (e.g., a pet or service animal). “A companion animal,” as used herein, refers to a pet and other domestic animal. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include rodents (e.g., rats, mice, guinea pigs, and hamsters), rabbits, or non-human primates.

In some embodiments, the agent to be administered to a subject is an agent that results in a decrease in the expression and/or activity of ARHGAP5 (e.g., in a cancer cell characterized by an ARHGAP35 alteration). In some embodiments, the agent to be administered to a subject is an agent that results in a decrease in the expression and/or activity of ARHGAP35 (e.g., in a cancer cell characterized by an ARHGAP5 alteration). As used herein, the term “expression” refers to the expression of a gene, namely ARHGAP35 or ARHGAP5, that results in the biosynthesis of a p190A or p190B protein, respectively. As used herein, the term “activity” refers to one or more activities of a protein, namely, p190A or p190B, and may include, but is not limited to, one or more enzymatic activities of p190A or p190B or one or more interactions between p190A or p190B and another molecule, such as a protein, a peptide, a nucleic acid, a lipid, a carbohydrate, or an ion. An agent described herein may reduce the expression or activity of ARHGAP35 or ARHGAP5, and may reduce both the expression and activity of ARHGAP35 or ARHGAP5. Those of ordinary skill in the art will readily recognize that an agent that reduces the expression of a given factor (e.g., a protein) will generally also reduce the activity of the factor by reducing the total number of molecules of the factor that are produced by a cell. An agent described herein may permanently (irreversibly) reduce the expression and/or activity of ARHGAP35 or ARHGAP5, or may transiently (reversibly) reduce the expression and/or activity of ARHGAP35 or ARHGAP5.

In some embodiments, the agent to be administered to a subject is an agent that reduces the expression and/or activity of ARHGAP35 or ARHGAP5. In some embodiments, the agent to be administered to a subject is an agent that reduces the expression and/or activity of ARHGAP5 in a cancer cell, e.g., in a cancer cell with decreased expression and/or activity of ARHGAP35 (e.g., as a result of an alteration in ARHGAP35). In some embodiments, the agent to be administered to a subject is an agent that reduces the expression and/or activity of ARHGAP35 in a cancer cell, e.g., a cancer cell with decreased expression and/or activity of ARHGAP5 (e.g., as a result of an alteration in ARHGAP5).

In some embodiments, the agent is an agent that reduces the expression and/or activity of ARHGAP5. In some embodiments, the agent reduces the expression and/or activity of human ARHGAP5 (e.g., NCBI Reference Sequence: NP_001025226.1; Gene ID: 394).

In some embodiments, the agent is an agent that reduces the expression and/or activity of ARHGAP35. In some embodiments, the agent reduces the expression and/or activity of human ARHGAP35 (e.g., NCBI Reference Sequence: NP_004482.4; Gene ID: 2909).

In some embodiments, the agent is an agent that inhibits the expression of ARHGAP5. In some embodiments, the agent is an agent that inhibits the expression of ARHGAP35. In some embodiments, the agent comprises a small interfering RNA (siRNA), a short hairpin RNA (shRNA), or an antisense oligonucleotide (ASO) that is complementary to a gene encoding the factor associated with myeloid cell activation. In some embodiments, the agent comprises a viral vector that encodes a siRNA or a shRNA that is complementary to a gene encoding ARHGAP5 (p190B). In some embodiments, the agent comprises a viral vector that encodes a siRNA or a shRNA that is complementary to a gene encoding ARHGAP35 (p190A). Examples of viral vectors known in the art that are suitable for delivery of siRNA or shRNA include, but are not limited to, lentiviral vectors and recombinant adeno-associated viral vectors (rAAV). In some embodiments, a siRNA, shRNA, or ASO that inhibits expression of ARHGAP5 (p190B) is at least 60%, at least 70%, at least 80%, at least 90%, at least 95%, at least 99%, or 100% complementary to a region of a gene encoding ARHGAP5 (p190B). In some embodiments, a siRNA, shRNA, or ASO that inhibits expression of ARHGAP35 (p190A) is at least 60%, at least 70%, at least 80%, at least 90%, at least 95%, at least 99%, or 100% complementary to a region of a gene encoding ARHGAP35 (p190A). In some embodiments, an agent that inhibits the expression of ARHGAP5 further reduces the overall activity of the p190B in a cell. In some embodiments, an agent that inhibits the expression of ARHGAP35 further reduces the overall activity of the p190A in a cell.

In some embodiments, the agent is an agent that inhibits the activity of ARHGAP5. In some embodiments, the agent is an agent that inhibits the activity of ARHGAP35. In some embodiments, the agent is a protein, a peptide, an aptamer, or a small molecule that inhibits the activity of ARHGAP5 (p190B), the meaning of each of which is well known to those of ordinary skill in the art. In some embodiments, the agent is a protein, a peptide, an aptamer, or a small molecule that inhibits the activity of ARHGAP35 (p190A). In some embodiments, the agent binds to (physically interacts with) ARHGAP5 (p190B). In some embodiments, the agent binds to (physically interacts with) ARHGAP35 (p190A). In some embodiments, the agent that inhibits the activity of ARHGAP35 or ARHGAP5 is a small molecular inhibitor.

In some embodiments, the subject is administered an effective amount of a single agent that results in reduced expression or activity of ARHGAP5 or ARHGAP35. In some embodiments, the subject is administered an effective amount of more than one agent (e.g., 2 agents or more) that results in reduced expression or activity of ARHGAP5 or ARHGAP35.

Conventional methods, known to those of ordinary skill in the art of medicine, can be used to administer the agent (or agents) to the subject, depending upon the type of disease to be treated (e.g., a cancer) or the site of the disease (e.g., an organ, a tissue, or a biological fluid). The agent (or agents) can be administered systemically (i.e., throughout the body) or locally (i.e., to one or more specific organs, tissues, or locations in the body). The agent (or agents) can also be administered via any conventional route, e.g., administered orally, parenterally, by inhalation spray, topically, rectally, nasally, buccally, vaginally, intraperitoneally, or via an implanted reservoir. The term “parenteral” as used herein includes subcutaneous, intracutaneous, intravenous, intramuscular, intraarticular, intraarterial, intrasynovial, intrasternal, intraperitoneal, intrathecal, intralesional, and intracranial injection or infusion techniques. In some embodiments, an agent described herein is administered via intravenous injection or infusion. In some embodiments, an agent described herein is administered intraocularly, for example, by an intraocular injection. In addition, an agent described herein may be administered to the subject via injectable depot routes of administration such as using 1-, 3-, or 6-month depot injectable or biodegradable materials and methods. In embodiments where more than one agent is administered to a subject, the agents may be administered by the same delivery route or by different delivery route. In embodiments where more than one agent is administered to a subject, the agents may be administered simultaneously (e.g., at the same time as part of the same administered composition (e.g., pharmaceutical composition), or as separate compositions (e.g., pharmaceutical compositions)). In embodiments where more than one agent is administered to a subject, the agents may be administered sequentially (e.g., at different times as separate compositions (e.g., pharmaceutical compositions)).

In some embodiments, an agent described herein is administered to the subject more than once. In some embodiments, an agent described herein is administered to the subject once per day, once per 2 days, once per 3 days, once per 4 days, once per 5 days, once per 6 days, once per week, once per 2 weeks, once per 3 weeks, once per month, once per 2 months, once per 3 months, once per 4 months, once per 6 months, once per 7 months, once per 8 months, once per 9 months, once per 10 months, once per 11 months, or once per year.

In some embodiments, the subject (e.g., a human patient) is a subject that has, is suspected of having, or is at risk of developing a cancer. In some embodiments, the subject (e.g., a human patient) is a subject that has, is suspected of having, or is at risk of developing a cancer having decreased expression or activity of ARHGAP35. In some embodiments, the cancer is a cancer in which ARHGAP35 mRNA expression is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue (e.g., control non-cancerous tissue, or non-cancerous tissue of the same subject). In some embodiments, the subject (e.g., a human patient) is a subject that has, is suspected of having, or is at risk of developing a cancer having decreased expression or activity of ARHGAP5. In some embodiments, the cancer is a cancer in which ARHGAP5 mRNA expression is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue (e.g., control non-cancerous tissue, or non-cancerous tissue of the same subject). In some embodiments, the cancer is a cancer in which activity of a p190A protein translated from an ARHGAP35 mRNA (e.g., an ARHGAP35 mRNA transcribed from an ARHGAP35 gene encoded by cells of the subject) is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue (e.g., control non-cancerous tissue, or non-cancerous tissue of the same subject). In some embodiments, the cancer is a cancer in which activity of a p190B protein translated from an ARHGAP5 mRNA (e.g., an ARHGAP5 mRNA transcribed from an ARHGAP5 gene encoded by cells of the subject) is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue (e.g., control non-cancerous tissue, or non-cancerous tissue of the same subject).

In some embodiments, specifically embodiments in which the subject is administered an effective amount of an agent that results in a decrease in the expression and/or activity of ARHGAP5, the cancer comprises at least one alteration of ARHGAP35. In some embodiments, the alteration of ARHGAP35 is a mutation in a gene encoding ARHGAP35. In some embodiments, the alteration of ARHGAP35 is a deletion of a gene encoding ARHGAP35. In some embodiments, the alteration of ARHGAP35 is an epigenetic modification in a gene encoding ARHGAP35.

In some embodiments, specifically embodiments in which the subject is administered an effective amount of an agent that results in a decrease in the expression and/or activity of ARHGAP35, the cancer comprises at least one alteration of ARHGAP5. In some embodiments, the alteration of ARHGAP5 is a mutation in a gene encoding ARHGAP5. In some embodiments, the alteration of ARHGAP5 is a deletion of a gene encoding ARHGAP5. In some embodiments, the alteration of ARHGAP5 is an epigenetic modification in a gene encoding ARHGAP5.

In some embodiments, the cancer is an oncogene-negative cancer (i.e., a cancer that is not known to be characterized by expression and/or activity of a known oncogene). In some embodiments, the cancer is a cancer selected from, but not limited to, a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer. In some embodiments, the cancer is an endometrial carcinoma, a uterine carcinosarcoma, a colon adenocarcinoma, a lung squamous carcinoma, a bladder cancer, a cervical carcinoma, or a stomach cancer. In some embodiments, the cancer is a metastatic cancer.

In some embodiments, the cancer is a cancer in which the Salvador-Warts-Hippo (SWH) pathway is inactivated. In some embodiments, the cancer is a cancer in which the level of SWH pathway activation in the cancer is decreased by up to 10%, up to 20%, up to 30%, up to 40%, up to 50%, up to 60%, up to 70%, up to 80%, up to 90%, up to 95%, up to 99%, or up to 100% as compared to the level of SWH activation in non-cancerous tissue (e.g., control non-cancerous tissue, or non-cancerous tissue of the same subject).

In some embodiments, the cancer is a cancer in which the level of activated Yes-associated protein (YAP) and/or Transcriptional coactivator with PDZ-binding motif (TAZ) is increased. In some embodiments, the cancer is a cancer in which the level of YAP and/or TAZ is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more, as compared to the level of YAP and/or TAZ in non-cancerous tissue (e.g., control non-cancerous tissue, or non-cancerous tissue of the same subject).

In some embodiments, the cancer is a cancer in which the level of activated Rho (Rho-GTP) is increased. In some embodiments, the cancer is a cancer in which the level of Rho-GTP is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more, as compared to the level of Rho-GTP in non-cancerous tissue (e.g., control non-cancerous tissue, or non-cancerous tissue of the same subject).

In some embodiments, the cancer is a cancer in which the level of contact inhibition of cell proliferation (CIP) is decreased. In some embodiments, the cancer is a cancer in which the level of CIP in the cancer is decreased by up to 10%, up to 20%, up to 30%, up to 40%, up to 50%, up to 60%, up to 70%, up to 80%, up to 90%, up to 95%, up to 99%, or up to 100% as compared to the level of CIP in non-cancerous tissue (e.g., control non-cancerous tissue, or non-cancerous tissue of the same subject).

In some embodiments, it is determined whether the cancer comprises one or more alterations of ARHGAP35 prior to administration of one or more agents that result in a decrease in expression and/or activity of ARHGAP5 to the subject. In some embodiments, the determination of one or more alterations of ARHGAP35 in the cancer comprises collecting a sample from the subject, sequencing the genome of one or more cancer cells present in the sample, and determining the presence of one or more alterations in the cancer. Methods for single cell sequencing, particularly in the context of cancer diagnosis and prognosis, are well known and routine to those of ordinary skill in the art, as well as methods for identifying genes of interest and determining if one or more alterations are present. In some embodiments, the sample comprises a tissue biopsy, blood sample, a serum sample, a plasma sample, a saliva sample, a sputum sample, a urine sample, a fecal sample, a lymphatic fluid sample, a synovial fluid sample, a cerebrospinal fluid sample, or an interstitial fluid sample. In some embodiments, a sample that is a tissue biopsy is a tumor biopsy. In some embodiments, the one or more alterations of ARHGAP35 comprise a mutation in a gene encoding ARHGAP35. In some embodiments, the one or more alterations of ARHGAP35 comprise a deletion of a gene encoding ARHGAP35. In some embodiments, the one or more alterations of ARHGAP35 comprise an epigenetic modification in a gene encoding ARHGAP35.

In some embodiments, it is determined whether the cancer comprises one or more alterations of ARHGAP5 prior to administration of one or more agents that result in a decrease in expression and/or activity of ARHGAP35 to the subject. In some embodiments, the determination of one or more alterations of ARHGAP5 in the cancer comprises collecting a sample from the subject, sequencing the genome of one or more cancer cells present in the sample, and determining the presence of one or more alterations in the cancer. In some embodiments, the sample comprises a tissue biopsy, blood sample, a serum sample, a plasma sample, a saliva sample, a sputum sample, a urine sample, a fecal sample, a lymphatic fluid sample, a synovial fluid sample, a cerebrospinal fluid sample, or an interstitial fluid sample. In some embodiments, a sample that is a tissue biopsy is a tumor biopsy. In some embodiments, the one or more alterations of ARHGAP5 comprise a mutation in a gene encoding ARHGAP5. In some embodiments, the one or more alterations of ARHGAP5 comprise a deletion of a gene encoding ARHGAP5. In some embodiments, the one or more alterations of ARHGAP5 comprise an epigenetic modification in a gene encoding ARHGAP5.

In some embodiments, the administration results in an increase in SWH pathway activation in the cancer, as compared to SWH pathway activation in the cancer prior to the administration. In some embodiments, the level of SWH pathway activation in the cancer is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more, as compared to SWH pathway activation in the cancer prior to the administration.

In some embodiments, the administration results in a decrease in the level of activated YAP and/or TAZ in the cancer, as compared to the level of activated YAP and/or TAZ in the cancer prior to the administration. In some embodiments, the level of activated YAP and/or TAZ in the cancer is decreased by up to 10%, up to 20%, up to 30%, up to 40%, up to 50%, up to 60%, up to 70%, up to 80%, up to 90%, up to 95%, up to 99%, or up to 100%, as compared to the level of activated YAP and/or TAZ in the cancer prior to the administration.

In some embodiments, the administration results in a decrease in the level of Rho-GTP in the cancer, as compared to the level of Rho-GTP in the cancer prior to the administration. In some embodiments, the level of Rho-GTP in the cancer is decreased by up to 10%, up to 20%, up to 30%, up to 40%, up to 50%, up to 60%, up to 70%, up to 80%, up to 90%, up to 95%, up to 99%, or up to 100%, as compared to the level of Rho-GTP in the cancer prior to the administration.

In some embodiments, the administration results in an increase of CIP activity in the cancer, as compared to CIP activity in the cancer prior to the administration. In some embodiments, the level of CIP activity in the cancer is increased by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 6-fold, up to 7-fold, up to 8-fold, up to 9-fold, or up to 10-fold or more, as compared to CIP activity in the cancer prior to the administration.

In some embodiments, the administration results in clearance of the cancer in the subject. In some embodiments, the administration results in clearance of up to 10%, up to 20%, up to 30%, up to 40%, up to 50%, up to 60%, up to 70%, up to 80%, up to 90%, up to 95%, up to 99%, or 100% of the cancer in one or more organs or tissues of the subject. In some embodiments, the administration results in clearance of up to 10%, up to 20%, up to 30%, up to 40%, up to 50%, up to 60%, up to 70%, up to 80%, up to 90%, up to 95%, up to 99%, or 100% of the cancer in the subject.

### Kits

Also encompassed by the present disclosure are kits (e.g., pharmaceutical packs), such as a kit for use in administering a composition or an agent (e.g., an agent that inhibits the expression or activity of ARHGAP5) described herein to a subject. The kits provided herein may comprise a pharmaceutical composition or an agent (e.g., an agent that inhibits the expression or activity of ARHGAP5) described herein and a container (e.g., a vial, ampule, bottle, syringe, and/or dispenser package, or other container suitable for storage and/or administration). In some embodiments, a kit provided herein may optionally further include a second container comprising a pharmaceutical excipient for dilution or suspension of a composition or an agent (e.g., an agent that inhibits the expression or activity of ARHGAP5) described herein, prior to administration to a subject. In some embodiments, a pharmaceutical composition or an agent (e.g., an agent that inhibits the expression or activity of ARHGAP5) described herein is provided in the first container and the second container are combined to form one dosage unit. In some embodiments, a kit provided herein may further optionally provide a device for administering a composition or an agent (e.g., an agent that inhibits the expression or activity of ARHGAP5) described herein to a subject (e.g., a syringe). In some embodiments, the device for administering a composition or an agent (e.g., an agent that inhibits the expression or activity of ARHGAP5) described herein and the container for storing the composition or an agent (e.g., an agent that inhibits the expression or activity of ARHGAP5) described herein are the same.

Thus, in one aspect, provided herein are kits including a first container comprising a composition or an agent (e.g., an agent that inhibits the expression or activity of ARHGAP5) described herein. In certain embodiments, the kits are useful for treating a disease (e.g., a cancer) in a subject in need thereof. In certain embodiments, the kits are useful for preventing a disease (e.g., a cancer) in a subject in need thereof.

In certain embodiments, a kit described herein further includes instructions for using the pharmaceutical composition or agent included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., a cancer) in a subject in need thereof. In certain embodiments, the kits and instructions provide for preventing a disease (e.g., a cancer) in a subject in need thereof. A kit described herein may include one or more additional pharmaceutical agents described herein as a separate composition or agent.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: ARHGAP35 (p190A) Functions as a Tumor Suppressor by Promoting Contact Inhibition of Cell Proliferation Through the Hippo Pathway

Genome-wide association studies (GWAS) have established ARHGAP35 as one of the 30-40 most significantly mutated genes in human tumor samples. It has further been determined that a region on chromosome 19 within which the ARHGAP35 gene is located ranks among the most frequently deleted regions in cancer. ARHGAP35 has been established as pan-cancer gene which is not linked to any particular type of cancer. Furthermore, in lung adenocarcinoma (LUAD) ARHGAP35 alterations are found predominantly in oncogene negative cancers. Such cancers exhibit a significantly higher number of alterations, thereby rendering them more difficult to model for functional studies. In spite of several follow-up studies, GWAS analyses have been unable to assign a role for ARHGAP35 in cancer beyond suggesting a tumor suppressor function.

ARHGAP35 encodes p190A RhoGAP (p190A), an enzyme that promotes GTP hydrolysis on Rho and Rac GTPases. However, p190A and its paralog p190B, which is encoded by ARHGAP5, are large, complex, multifunctional proteins, comprising a Ras-like domain, four FF domains, two pseudo-GAP domains, and an active GAP domain, in addition to sequences of unknown function. These domains influence GAP activity, for instance, via interaction with Rnd and Rac proteins to control Rho-GTP levels and downstream effector signaling. The p190 proteins also exert scaffolding functions which is exemplified by the p190A-mediated sequestration of the transcription factor TFII-I in the cytoplasm.

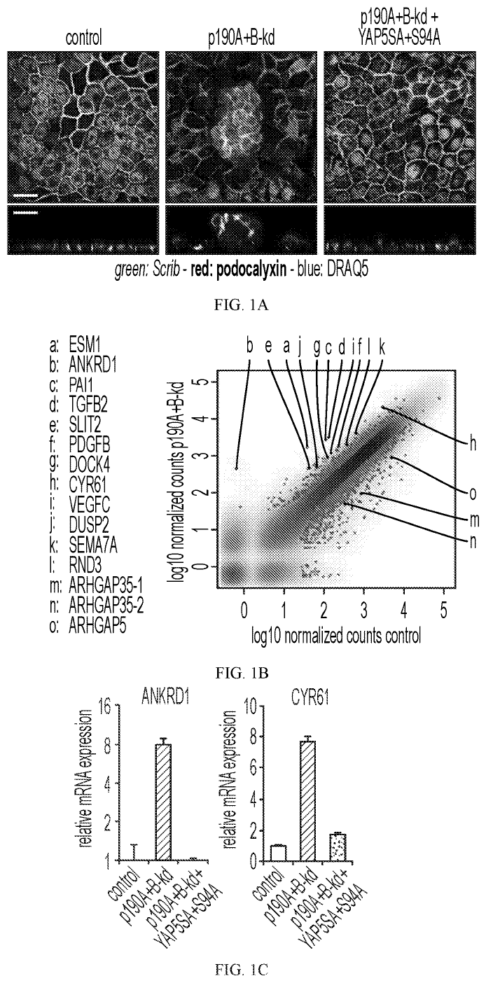

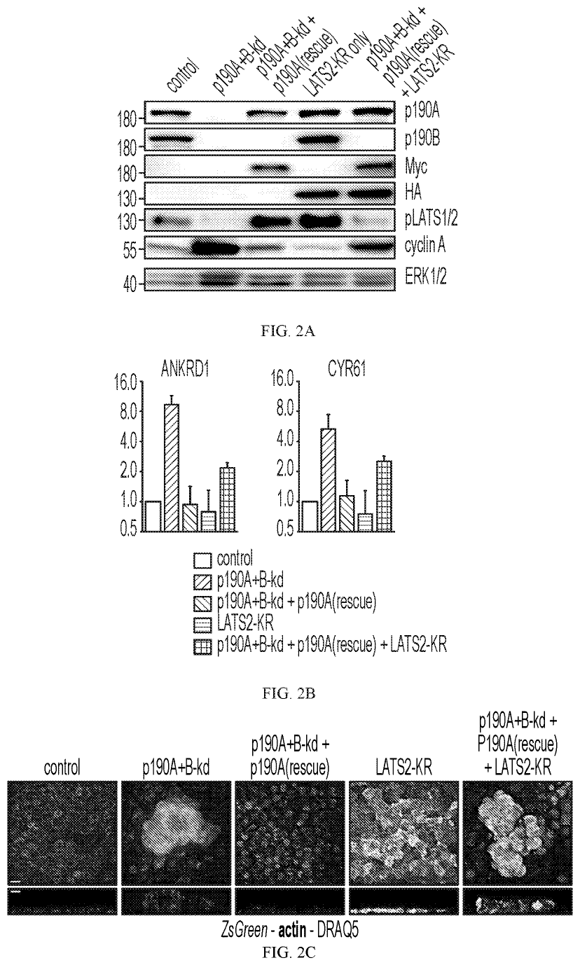

The mechanisms by which p190A and p190B modulate oncogenic capacities in epithelial cells were thus explored, particularly given that the vast majority of human cancers are adenocarcinomas such as LUAD. To investigate a role for p190A in epithelial cell transformation, Madin-Darby canine kidney (MDCK) cells were generated with inducible knockdown of p190A and/or p190B. MDCK cells are immortalized, non-oncogenic, diploid cells with highly preserved epithelial characteristics. Upon knockdown of p190A together with p190B, it was noted that MDCK cells failed to undergo contact inhibition of cell proliferation (CIP) (FIG. 1A). Next, transcriptome analyses showed that approximately one-third of the 30 most strongly induced genes in cells with p190A and p190B knockdown were target genes of the proto-oncogenic transcriptional co-activator YAP (FIG. 1B). Subsequent expression studies revealed that p190A and p190B promote CIP by repressing Yes-associated protein (YAP) function which, along with its paralog transcriptional coactivator with PDZ-binding motif (TAZ), acts as a transcriptional activator that is overexpressed in a range of cancers. The transcriptional activity of YAP is inhibited by the canonical Salvador-Warts-Hippo (Hippo) pathway and promoted by mechano-transduction. The operational definition of the canonical Hippo pathway is dependency on LATS1/2 kinases, while mechano-transduction is defined as ROCK-dependent. It was determined that p190A attenuates transcription of YAP target genes and promotes CIP via activation of LATS kinases (FIGS. 2A-2C). In was further determined that Rho-ROCK signaling is necessary for YAP function and perturbation of CIP in cells depleted of p190A and p190B. However, overexpression of highly active RhoA (Q63L) in MDCK cells does not evoke a YAP response, nor does it perturb CIP. These results suggest that there are scaffolding functions of p190A which are essential to activate LATS kinases, repress YAP-mediated gene transcription and promote CIP in epithelial cells.

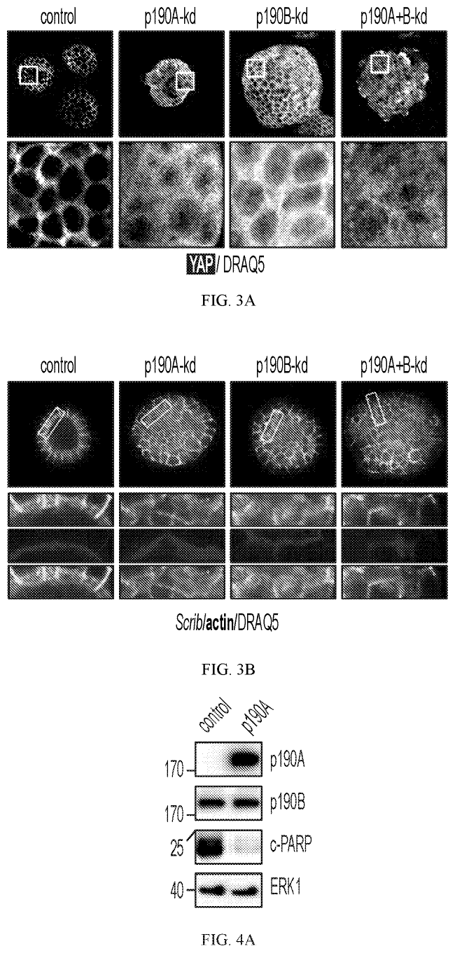

Importantly, when MDCK cells were cultured in 3D Matrigel matrices, depletion of p190A or p190B alone is sufficient to promote YAP translocation to the nucleus and perturb CIP (FIGS. 3A and 3B). Hence, combined loss of p190A and p190B may be neither necessary for activation of the Hippo pathway nor effective in promoting epithelial oncogenesis. Knockdown of p190B in addition to p190A in MDCK cells is likely required to enhance mechanical stress, thus lowering the threshold for YAP activation. Subsequently, in the presence of a stronger cell-matrix interaction, spheroids lacking both p190A and p190B expression were observed to have numerous fragmented nuclei, suggesting that depletion of both genes together is deleterious.

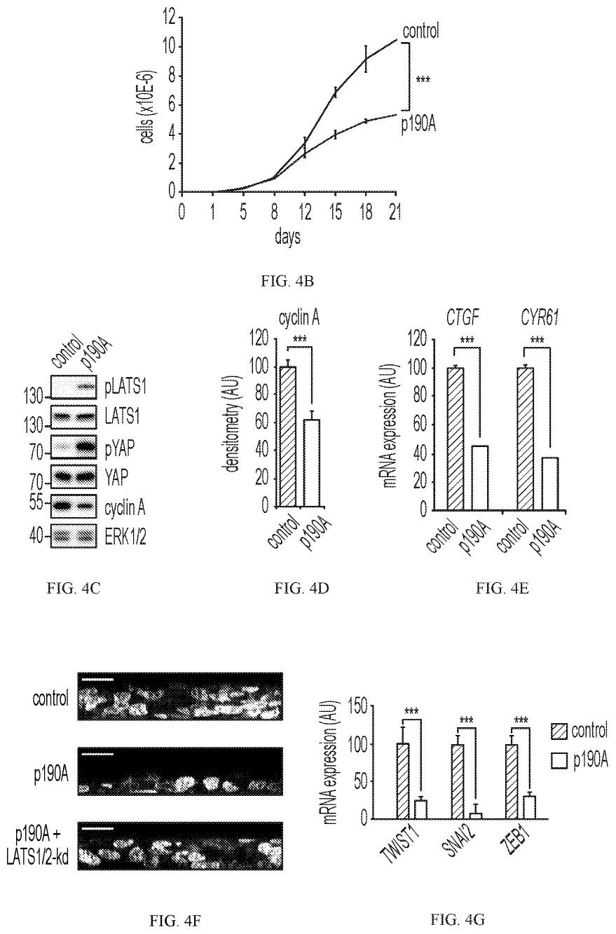

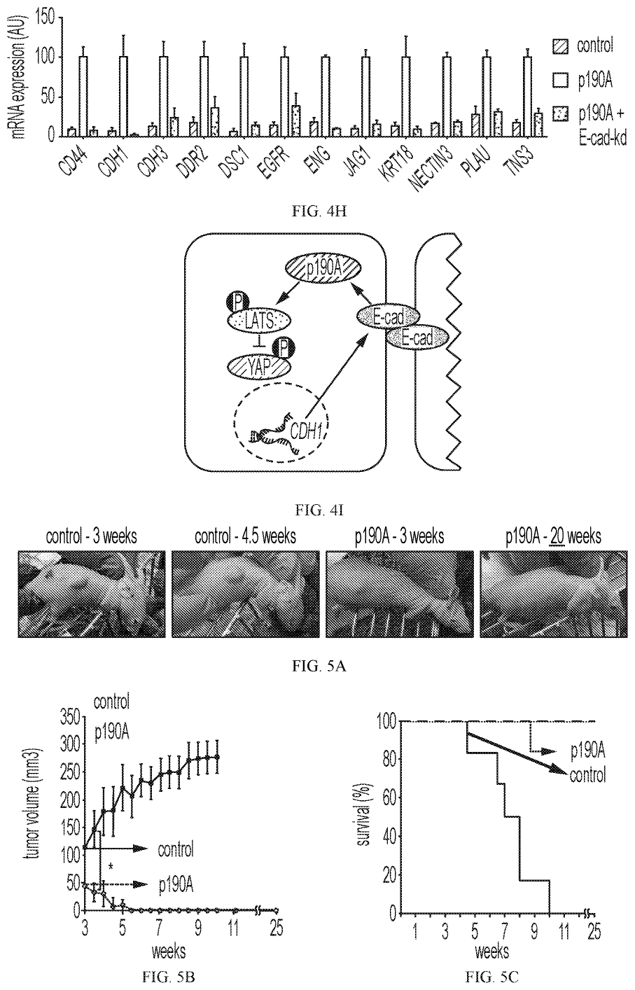

Despite the range of ARHGAP35 alterations that occur in human cancer, numerous co-occurring alterations in other genes makes it challenging to interpret the exact role of ARHGAP35 in cancer. To better evaluate this role, cancer cell lines harboring an ARHGAP35 alteration were developed and it the effect of exogenous p190A in the cancer was assessed. To this end, the Cancer Cell Line Encyclopedia (CCLE) was examined and NCI-H226 (H226) and NCI-H661 (H661) cells were identified as having low or no p190A expression (FIG. 4A). It was then determined that expression of p190A in these cell lines activates the Hippo pathway and promotes CIP (FIGS. 4B-4E). Strikingly, upon reconstitution with p190A, H661 cells underwent morphological differentiation into cuboidal cells that grow in monolayers, as opposed control cells which exhibit extensive multilayering (FIG. 4F). These results validate previous finding in MDCK cells, despite the apparent differences in these cell types. Using mRNA sequencing (mRNA-seq) analysis, it was further uncovered that p190A represses expression of TWIST1, SNAI2, and ZEBI to elicit mesenchymal-to-epithelial transition (MET) (FIG. 4G). E-cadherin was found to be required for expression of epithelial differentiation genes (FIG. 4H). Given that E-cadherin activates the canonical Hippo pathway, a role for p190A in activation of LATS kinases downstream of E-cadherin was further assessed in both H661 cells and in BRCA MDA-MB-231 cells. The results revealed that p190A is necessary for E-cadherin to activate LATS kinases and CIP. In turn, it was determined that LATS kinases were required for p190A-induced expression of E-cadherin. Collectively, these data are consistent with a model whereby p190A establishes a feed-forward cycle to activate the Hippo pathway, induce expression of E-cadherin, which in turn enhances LATS activation to repress YAP-mediated gene transcription and thereby promote CIP (FIG. 4I).

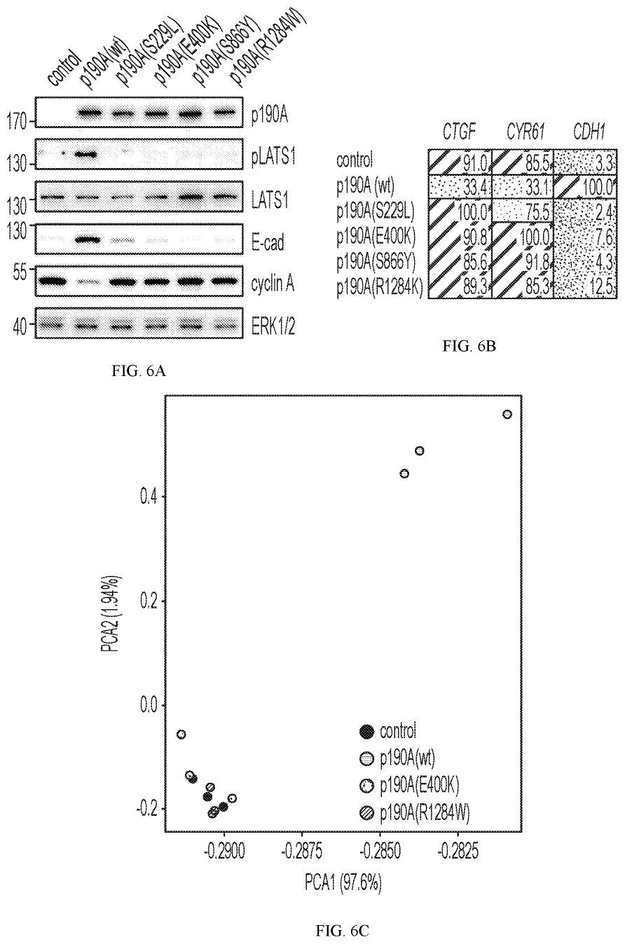

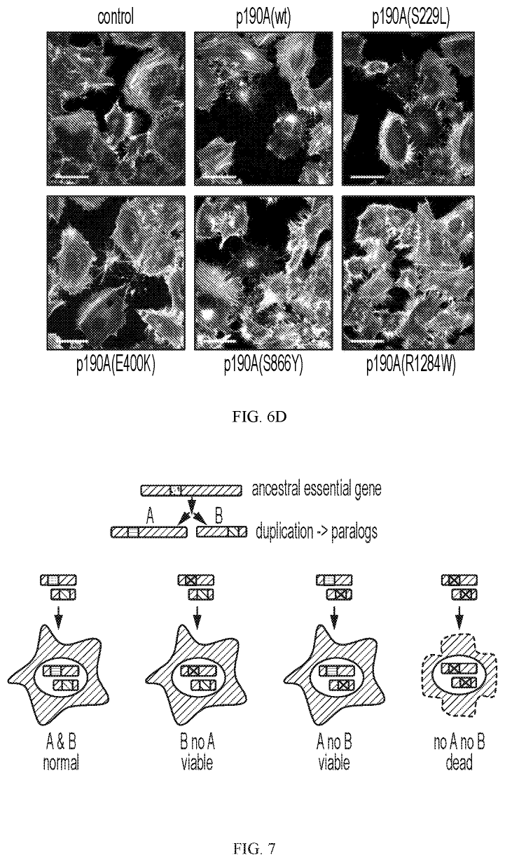

To formally test a role for p190A as a tumor suppressor, severe combined immunodeficient (SCID) mice were injected with control H661 cells or H661-p190A cells. Mice injected with control cells developed tumors that necessitated euthanasia when tumors reached 8 mm in diameter after 4.5-10 weeks (FIG. 5A). At longer timeframes, large intraabdominal metastases were observed. In striking contrast, mice injected with H661-p190A cells developed small tumors that peaked in size after approximately 3 weeks and subsequently regressed completely. These mice were carefully monitored for 25 weeks without finding any signs of recurrence either clinically or by autopsy (FIGS. 5B and 5C). Mice injected with cells expressing p190A forms harboring recurrent cancer mutations, including the R1284W mutation of the critical arginine finger residue in the GAP domain, exhibited intermediate phenotypes in vivo. In vitro, it was further determined that p190A forms harboring recurrent cancer mutations failed to activate LATS kinases, induce CIP, promote MET, or elicit transcriptomic alterations, as observed for wild type p190A (FIGS. 6A-6C). Importantly, several of these mutants retain full GAP activity (FIG. 6D). Taken together, these results indicate the mechanisms underlying the tumor suppressor capacities of ARHGAP35 encoding p190A.

### Example 2: Paralog Lethality can be Induced by Targeting ARHGAP5 (p190B) in Cancer Cells with ARHGAP35 (p190A) Alteration

Paralog lethality refers to a phenomenon that occurs when a cell requires one paralog for cell survival when other paralogs are lost due to a function-ablating alteration (FIG. 7). Paralog lethality could potentially be used to selectively eliminate cancer cells, as the viability of non-cancer cells expressing at least one paralog should be unaffected by the treatment. Given the results of the studies described in Example 1, it was considered that p190B, which is encoded by ARHGAP5, represents a paralog lethality target in cancer cells that have low or no expression of p190A. ARHGAP5 is found in vertebrates, but not invertebrates, and evolved by gene duplication from ARHGAP35, which is present in vertebrates and invertebrates alike. Mice that are null for Arhgap5 (p190B^{-/-}) or Arhgap35 (p190A⁻/−) die shortly after birth, while the double knockout is embryonically lethal. However, both p190A^{-/-} and p190B^{-/-} mouse embryonic fibroblasts (MEFs) can be cultured no differently than those from wild type littermates, suggesting that embryonic lethality occurs due to a developmental defect. Similarly, MDCK cells depleted of p190A or p190B individually exhibit preserved epithelial characteristics in 2D culture and remain viable when grown in 3D Matrigel matrices.

Paralog buffering refers to expression of a single paralog that is sufficient to execute specific cellular functions. Given that it may be advantageous to target capacities other than cell survival in p190A-deficient cancer cells, paralog buffering between p190A and p190B in epithelial cells was defined further. In particular the effect of paralog buffering between p190A and p190B on relevant oncogenic capacities was examined, particularly cell migration, cell proliferation, anoikis, cell-cell competition, and tumorigenesis.

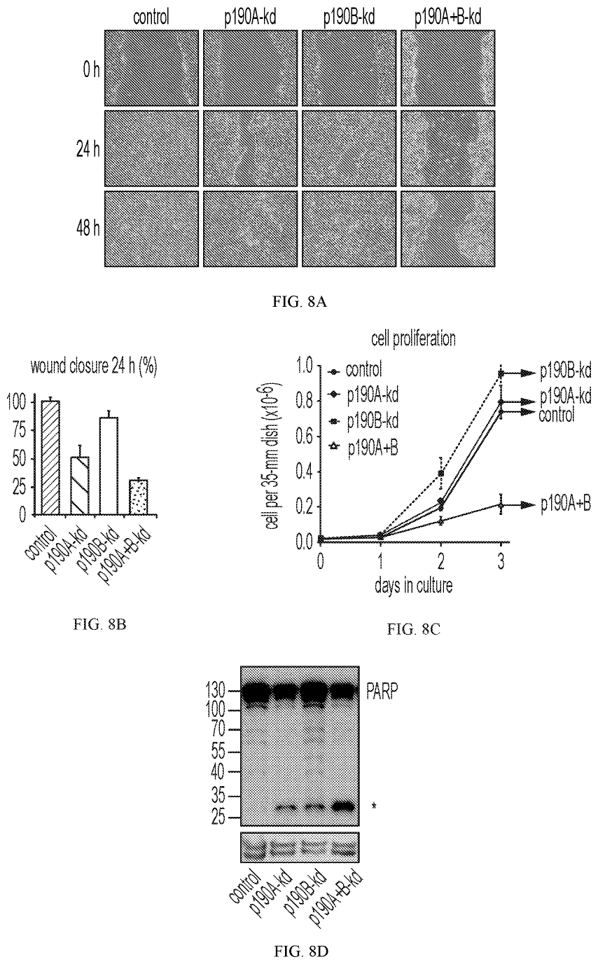

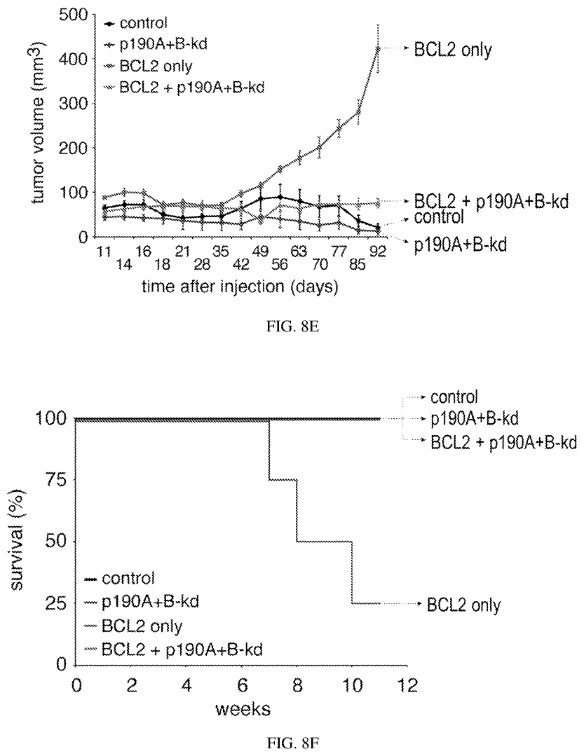

In vitro scratch wound assays were conducted with confluent MDCK cell monolayers in order to assess the rate at which the wounds close between cells depleted of p190A and/or p190B, relative to control cells. As expected, while MDCK cells depleted of p190A or p190B expression closed the wound within 48 hours, similar to control cells, those that were depleted of both p190A and p190B expression displayed a significant delay in wound closure (FIGS. 8A and 8B). These results indicate that cells lacking p190A and p190B have impaired cell motility, polarization, and/or proliferation.

To specifically assess defects in cell proliferation, a cell growth assay was conducted. Control MDCK cells, MDCK cells lacking p190A or p190B expression, and MDCK cells lacking both p190A and p190B expression were plated in 6-well plates at a density of 5×10⁴ cells per well and cultured for several days. On each day over the course of the experiment, cells from a minimum of three wells per condition were harvested and quantified. After three days, a proliferation defect was observed in cells without p190A and p190B expression, while cells with p190A or p190B expression did not grow at a significantly slower rate than control cells (FIG. 8C). These results indicate that p190A and p190B exhibit a paralog buffering effect on cellular growth rate.

Next, the effect of p190A and p190B upon anoikis was examined. Anoikis is defined as apoptosis resulting from cadherin-engagement in the absence of cell-substratum interaction. Resistance to anoikis is thought to be essential for cancer cell survival during metastasis, by allowing metastatic cancer cells to separate from tumors and spread via blood and lymphatic vessels, as well as through pleural and peritoneal cavities before establishing new tumors. To assay anoikis, MDCK cells depleted of p190A and/or p190B were harvested by trypsinization and 2×10⁶ cells were plated into each well of 24-well ultra-low adhesion plates. The cells were cultured for up to 8 hours in suspension, pelleted by centrifugation, and lysed directly into Laemmli buffer containing 6 M urea. Whole cell lysates were then subjected to sodium dodecyl sulphate polyacrylamide gel electrophoresis (SDS-PAGE), after which full-length poly(ADP-ribose) polymerase-1 (PARP) and cleaved PARP were detected by immunoblotting and quantified by densitometry. MDCK cells lacking both p190A and p190B expression exhibited an enhanced quantity of cleaved PARP, an indicator of apoptosis, as compared to control cells and cells lacking expression of p190A or p190B (FIG. 8D). These results indicate increased cell death occurring in MDCK cells in the absence of p190A and p190B function, suggesting that these paralogs could be targeted in human cancer cells.

To test whether these paralogs could be targeted in human cancer cells, cancerous MDCK cells were generated and tested in vivo using a mouse xenograft model. Mice were inoculated with either control MDCK cells or MDCK cells engineered to express a B-cell lymphoma 2 (BCL2) oncogene, either in the presence or absence of p190A and p190B knockdown. The volume of tumors was then monitored for up to 92 days after inoculation. BCL2+ MDCK tumors grew steadily over the duration of the experiment, while MDCK tumors with only p190A and p190B knockdown grew similarly to tumors of control cells (FIG. 8E). Remarkably, BCL2+ MDCK tumors lacking p190A and p190B expression also grew similarly to tumors of control cells, despite expressing the potent BCL2 oncogene. Moreover, all mice with BCL2+ MDCK tumors that lacked p190A and p190B expression survived over the course of the experiment (FIG. 8F). These results provide a clear proof of principle that paralog lethality can be achieved in cancer cells by targeting the function of p190A and p190B, and that this strategy confers a substantial therapeutic benefit.

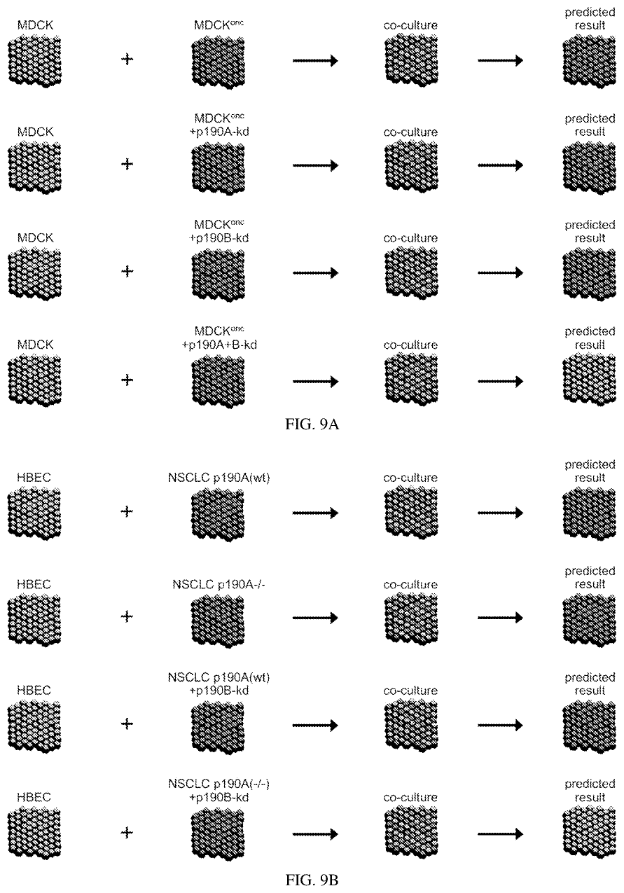

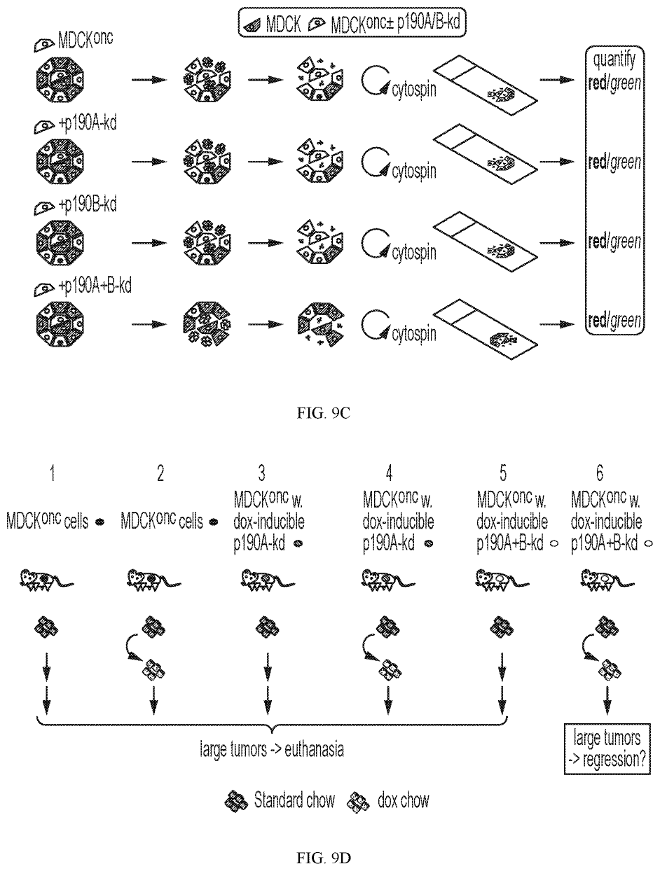

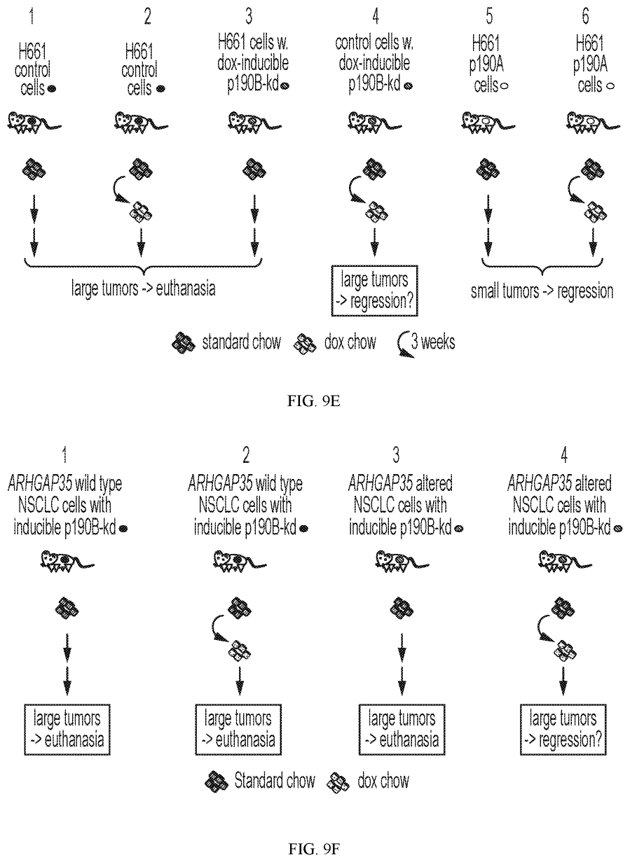

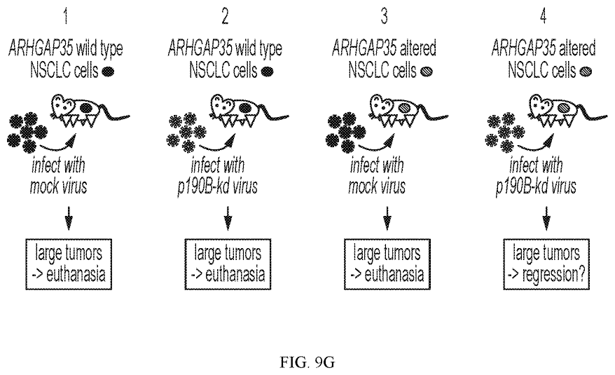

Additional techniques are also suitable for evaluating paralog lethality of p190A and p190B in cancer cells. First, MDCK cells may be tested in a co-culture assay comprising control MDCK cells and MDCK cells that express a known oncogene (e.g., BCL2), either in the presence or absence of p190A and/or p190B knockdown (FIG. 9A). Although oncogene-expressing cells are expected to outcompete control cells in the presence of p190A or p190B knockdown, knockdown of both p190A and p190B is expected to result in paralog lethality of the oncogene-expressing cells. Techniques such as genomic sequencing or fluorescent markers may be used to verify the proportion of resulting cells in the population that are control cells or oncogene-expressing cells. A similar assay may be performed by using human cells, in which co-cultures are assayed between primary human bronchial epithelial cells (HBEC) and human non-small cell lung cancer (NSCLC) cells lacking expression of p190A and/or p190B (FIG. 9B). Similarly, NSCLC cells lacking expression of p190A and p190B are expected to be outcompeted as a result of paralog lethality, producing a population of cells that is primarily composed of HBEC control cells. Additionally, co-cultures of control MDCK cells and oncogene-expressing MDCK cells (or alternately HBEC and NSCLC cells) may be assayed using an anoikis slide assay in which apoptosis of fluorescently labeled cells is examined under various conditions (FIG. 9C). As above, oncogene-expressing MDCK cells (or NSCLC cells) lacking expression of p190A and p190B are expected to undergo anoikis and die as a results of paralog lethality, while control cells labeled with a different fluorophore are expected to survive and become dominant in the cell population.

Other techniques may also be used to further assess paralog lethality of p190A and p190B in vivo. For example, mice may be inoculated with either oncogene-expressing MDCK cells, MDCK cells with doxycycline inducible knockdown of p190A, or MDCK cells with doxycycline inducible knockdown of p190A and p190B (FIG. 9D). Inoculated mice can then be fed either standard mouse chow until tumors form, and then optionally transitioned to a doxycycline-treated mouse chow to induce knockdown of p190A or p190A and p190B. Although mice are expected to develop large tumors that necessitate euthanasia under most conditions, mice with doxycycline induced knockdown of p190A and p190B are expected to exhibit regression of tumors due to paralog lethality, somewhat similarly to mice that were previously inoculated with H661 tumors in which p190A tumor suppression was reconstituted (FIGS. 5A-5C). A similar assay may also be conducted by inoculating mice with H661 control cells and H661 cells with doxycycline inducible knockdown of p190B (FIG. 9E). While mice inoculated with H661 control cells are expected to develop tumors necessitating euthanasia, those in which p190B is knocked down using doxycycline are expected to exhibit regression of tumors, due to the low level of p190A expression in H661 cells. These experiments may be conducted similarly using human cancer cells, such as NSCLC cells, with or without a known ARHGAP35 alteration and doxycycline inducible knockdown of p190B (FIG. 9F).

The effect of agents for reducing p190B expression in cancer cells with an ARHGAP35 alteration may also be explored using an in vivo model. For example, mice may be inoculated with NSCLC cells encoding either wild-type ARHGAP35 or an ARHGAP35 alteration, and then administered a mock agent or an agent capable of reducing expression of p190B (FIG. 9G). Such an agent may comprise an agent that inhibits p190B function, such as a small molecular inhibitor, or an agent the prevents translation of p190B, such as a small interfering RNA (siRNA), short hairpin RNA (shRNA), an antisense oligonucleotide (ASO), or another agent capable of mediating RNA interference (RNAi) of ARHGAP5 mRNA transcripts. Such agents may be delivered alone or in the presence of one or more delivery agents, or can be delivered in the form of a viral vector encoding an agent suitable for mediating RNAi. For example, mice bearing NSCLC tumors with and ARHGAP35 alteration may be administered a lentiviral or recombinant adeno-associated viral (rAAV) vector encoding a siRNA or shRNA that specifically targets ARHGAP5. A viral vector may be selected for tropism toward a particular tissue. For example, a vector (e.g., a rAAV) may be used to preferentially deliver a siRNA or shRNA targeting ARHGAP5 to lung epithelia in order to assess treatment of NSCLC tumors in the lung.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one member of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating a cancer in a subject, the method comprising administering to a subject in need thereof an effective amount of an agent that results in a decrease in the expression or activity of ARHGAP5, wherein the cancer is a cancer in which ARHGAP35 expression or activity is decreased.

2. The method of claim 1, wherein the agent inhibits expression of ARHGAP5 in the cancer.

3. The method of claim 2, wherein the agent comprises a small interfering RNA (siRNA), a short hairpin RNA (shRNA), or an antisense oligonucleotide (ASO) that is complementary to an ARHGAP5 mRNA expressed in the cancer.

4. The method of claim 3, wherein the agent comprises a viral vector that encodes a siRNA or a shRNA that is complementary to an ARHGAP5 mRNA expressed in the cancer.

5. The method of claim 4, wherein the viral vector is a lentiviral vector or a recombinant adeno-associated viral (rAAV) vector.

6. The method of any one of claims 3-5, wherein the siRNA, shRNA, or ASO is at least 60%, at least 70%, at least 80%, at least 90%, at least 95%, at least 99%, or 100% complementary to a region of an ARHGAP5 mRNA expressed in the cancer.

7. The method of claim 1, wherein the agent inhibits the activity and/or stability of a p190B protein translated from an ARHGAP5 mRNA expressed in the cancer.

8. The method of claim 7, wherein the agent comprises a small molecular inhibitor.

9. The method of any one of claims 1-8, wherein the cancer is a cancer in which ARHGAP35 mRNA expression is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue.

10. The method of any one of claims 1-9, wherein the cancer is a cancer in which activity of a p190A protein translated from an ARHGAP35 mRNA is decreased by up to 10%, up to 20%, up to 30%, up to 40%, up to 50%, up to 60%, up to 70%, up to 80%, up to 90%, up to 95%, up to 99%, or up to 100% as compared to non-cancerous tissue.

11. The method of any one of claims 1-10, wherein the cancer comprises at least one alteration of ARHGAP35.

12. The method of claim 11, wherein the alteration of ARHGAP35 is a mutation in a gene encoding ARHGAP35.

13. The method of claim 11, wherein the alteration of ARHGAP35 is a deletion of a gene encoding ARHGAP35.

14. The method of claim 11, wherein the alteration of ARHGAP35 is an epigenetic modification in a gene encoding ARHGAP35.

15. The method of any one of claims 1-14, wherein the cancer is an oncogene-negative cancer.

16. The method of any one of claims 1-15, wherein the cancer is a hematological cancer, a lung cancer, a breast cancer, a brain cancer, a gastrointestinal cancer, a liver cancer, a kidney cancer, a bladder cancer, a pancreatic cancer, an ovarian cancer, a testicular cancer, a prostate cancer, an endometrial cancer, a muscle cancer, a bone cancer, a neuroendocrine cancer, a connective tissue cancer, a head or neck cancer, or a skin cancer.

17. The method of claim 16, wherein the cancer is selected from endometrial carcinoma, uterine carcinosarcoma, colon adenocarcinoma, lung squamous carcinoma, bladder cancer, cervical carcinoma, or stomach cancer.

18. The method of any one of claims 1-17, wherein the cancer is a metastatic cancer.

19. The method of any one of claims 1-18, wherein the Salvador-Warts-Hippo (SWH) pathway is inactivated in the cancer.

20. The method of any one of claims 1-19, wherein the level of activated Yes-associated protein (YAP) and/or Transcriptional coactivator with PDZ-binding motif (TAZ) is increased in the cancer, as compared to non-cancerous tissue.

21. The method of any one of claims 1-20, wherein the level of activated Rho (Rho-GTP) is increased in the cancer, as compared to non-cancerous tissue.

22. The method of any one of claims 19-21, wherein contact inhibition of cell proliferation (CIP) activity is decreased in the cancer, as compared to non-cancerous tissue.

23. The method of any one of claims 1-22, wherein the subject is a mammal.

24. The method of claim 23, wherein the subject is a human.

25. The method of any one of claims 1-24, further comprising determining if the cancer comprises one or more alterations of ARHGAP35 prior to administration of the agent.

26. The method of claim 25, wherein the determination comprises:
(a) collecting a sample from the subject;
(b) sequencing the genome of one or more cancer cells present in the sample; and
(c) determining the presence of one or more alterations of ARHGAP35 in the cancer.

27. The method of claim 26, wherein the sample comprises a tissue biopsy, blood sample, a serum sample, a plasma sample, a saliva sample, a sputum sample, a urine sample, a fecal sample, a lymphatic fluid sample, a synovial fluid sample, a cerebrospinal fluid sample, or an interstitial fluid sample.

28. The method of claim 27, wherein the tissue biopsy is a tumor biopsy.

29. The method of any one of claims 21-28, wherein the one or more alterations of ARHGAP35 comprise a mutation in a gene encoding ARHGAP35.

30. The method of any one of claims 21-28, wherein the one or more alterations of ARHGAP35 comprise a deletion of a gene encoding ARHGAP35.

31. The method of any one of claims 21-28, wherein the one or more alterations of ARHGAP35 comprise an epigenetic modification in a gene encoding ARHGAP35.

32. The method of any one of claims 1-31, wherein the administration occurs systemically or locally.

33. The method of claim 32, wherein the administration occurs via injection.

34. The method of claim 33, wherein the injection is intravenous or intratumoral injection.

35. The method of any one of claims 1-34, wherein the administration occurs more than once.

36. The method of claim 35, wherein the administration occurs between once per week and once per six months.

37. The method of any one of claims 1-36, wherein the administration results in an increase in SWH pathway activation in the cancer, as compared to SWH pathway activation in the cancer prior to the administration.

38. The method of any one of claims 1-37, wherein the administration results in a decrease in the level of activated YAP and/or TAZ in the cancer, as compared to the level of activated YAP and/or TAZ in the cancer prior to the administration.

39. The method of any one of claims 1-38, wherein the administration results in an increase of CIP activity in the cancer, as compared to CIP activity in the cancer prior to the administration.

40. The method of any one of claims 1-39, wherein the administration results in clearance of the cancer in the subject.

41. The method of claim 40, wherein the administration results in clearance of up to 10%, up to 20%, up to 30%, up to 40%, up to 50%, up to 60%, up to 70%, up to 80%, up to 90%, up to 95%, up to 99%, or 100% of the cancer in the subject.

42. A method of treating a cancer in a subject, the method comprising administering to a subject in need thereof an effective amount of an agent that results in a decrease in the expression or activity of ARHGAP35, wherein the cancer is a cancer in which ARHGAP5 expression or activity is decreased.
